(19) United States
(12) Patent Application Publication
(10) Pub. No.: US 2025/0009421 A1
(43) Pub. Date: Jan. 9, 2025

(54) DEVICES, SYSTEMS, AND METHODS FOR TUNNELING BETWEEN TISSUE LAYERS

(71) Applicant: BOSTON SCIENTIFIC SCIMED, INC., MAPLE GROVE, MN (US)

(72) Inventors: Austin Grant Johnson, Worcester, MA (US); Barry Weitzner, Acton, MA (US); Gary A. Jordan, Litchfield, NH (US)

(73) Assignee: BOSTON SCIENTIFIC SCIMED, INC., MAPLE GROVE, MN (US)

(21) Appl. No.: 18/763,049

(22) Filed: Jul. 3, 2024

Related U.S. Application Data
(60) Provisional application No. 63/525,221, filed on Jul. 6, 2023.

(51) Int. Cl. A61B 18/14; A61B 17/32; A61B 18/00; A61M 25/10

(52) U.S. Cl. CPC A61B 18/1492; A61B 2017/320048; A61B 2017/320056; A61B 2018/00601; A61B 2018/0091; A61M 2025/1015; A61M 25/1018; A61M 2025/1081

(57) ABSTRACT

An assembly and/or system having a cutting device configured to cut tissue, and an inflatable device configured for insertion into the cut tissue to further separate the cut tissue. The inflatable device may be wedge shaped to separate the cut tissue upon insertion into the cut. The inflatable device is inflatable and otherwise adjustable to separate the tissue to a medically indicated extent. For instance, the inflatable device may be moved axially with respect to the cutting device; and/or may be sheathed or unsheathed to adjust an exposed inflated length thereof; and/or may include two or more separately inflatable elements. The inflatable device thus may be used to achieve blunt tissue dissection after an initial cut is made into the tissue with a sharper instrument.

### CROSS-REFERENCE TO RELATED APPLICATIONS

This application claims the benefit of priority under 35 U.S.C. § 119 to U.S. Provisional Application No. 63/525,221, filed Jul. 6, 2023, the entire disclosure of which is hereby incorporated by reference herein for all purposes.

### FIELD

The present disclosure relates generally to the field of medical devices for accessing spaces within anatomical structures. In particular, the present disclosure relates to medical devices, systems, and methods for cutting or tunneling in and/or between tissue layers of an anatomical structure.

### BACKGROUND

Various devices, assemblies, systems, methods, techniques, etc., exist for cutting, dissecting, resecting, excising, etc., anatomical structures (e.g., biological tissue, including lesions, etc.). The configurations of the cutting device (which may include a blade, needle, laser, or other structure capable of cutting anatomical structures) may be selected based on the particular procedure to be performed. The procedures may be performed using open surgery (accessing the interior of a patient's body by cutting open the body) or minimally invasive surgery (e.g., percutaneously, laparosopically, endoscopically, etc.). One endoscopic procedure is third space endoscopy (also known as submucosal endoscopy), which may access deeper layers of tissue within the body (e.g., the gastrointestinal (GI) tract) by tunneling into the tissue, such as between structurally differentiated layers of tissue. For instance, in the GI system, tunneling may be performed in the submucosal space with an endoscope, without compromising the integrity of the overlying mucosa. Typically, a fluid (e.g., saline) is injected at the treatment site to elevate/lift the tissue (to create a “bleb”) to facilitate cutting of the tissue, such as with a knife. For instance, a lifting agent may be injected into the submucosal layer to separate the mucosal layer from the muscularis layer. A cutting knife may then be used to cut through the submucosa. While tunneling, the medical scope (e.g., gastroscope) may have a tapered distal cap mounted over the distal end thereof to help pass the scope through the tissue at the target site. Blunt tissue dissection may be performed using a mechanical force to separate tissue layers. In some aspects, a scope cap may provide a form of blunt tissue dissection as the scope tapers through the tissue. However, cutting instruments, such as electrosurgical knives, typically are predominantly used to tunnel through tissue in third space endoscopic procedures. Typically, the cutting instrument is used to make multiple small cuts, typically sequentially, to create a desired separation of tissue gradually, such as to reduce the risk of creating too large a cut in the tissue. Safer and more efficient tunneling devices, systems, methods, etc., would be welcome in the field. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a tissue tunneling system includes a control handle at the proximal end thereof; an elongate delivery member extending distally from the control handle; a cutting device operatively associated with the elongate delivery member for delivery at the distal end of the tissue tunneling system; and an inflatable device operatively associated with the distal end of the tissue tunneling system.

In some aspects, the inflatable device is operatively associated with the distal end of the tissue tunneling system to be delivered to a target site proximal to the cutting device.

In some aspects, the inflatable device is axially movable with respect to the cutting device.

In some aspects, the inflatable device is operatively associated with a tubular elongate member axially shiftable with respect to the elongate delivery member. In some aspects, the tubular elongate member defines a lumen therethrough in fluid communication with the inflatable device to deliver an inflation medium to the inflatable device to selectively expand the inflatable device. In some aspects, the tubular elongate member defines a lumen therethrough and the elongate delivery member is insertable and axially translatable through the lumen of the tubular elongate member to move the cutting device and the inflatable device with respect to each other.

In some aspects, in addition to being inflatable, the configuration of the inflatable device is adjustable.

In some aspects, the system further includes a sheath defining a lumen therethrough sized for the inflatable device to fit therein, the sheath advanceable and retractable with respect to the inflatable device to extend over or to unsheathe the inflatable device.

In some aspects, the inflatable device includes two or more inflatable elements.

In some aspects, the control handle includes a first lumen for injection of fluid through a lumen defined through the cutting device, and a second lumen for injection of inflation fluid into the inflatable device.

In accordance with various principles of the present disclosure, a tissue tunneling assembly includes a cutting device at a distal end of the tissue tunneling assembly; and an inflatable device positioned proximal to the cutting device.

In some aspects, the inflatable device is axially movable with respect to the cutting device.

In some aspects, the assembly further includes a sheath with a lumen therethrough. In some aspects, the sheath is axially movable with respect to the inflatable device, and the inflatable device and the lumen are sized for the inflatable device to be selectively fully or partially sheathed within the lumen, partially extended and inflated outside the lumen, or fully extended and inflated outside the lumen.

In some aspects, the inflatable device comprises two or more separate independently inflatable elements.

In some aspects, the cutting device is an electrosurgical knife.

In accordance with various principles of the present disclosure, a method of tunneling into tissue includes advancing a tissue tunneling assembly with a cutting device to a target site; cutting tissue with the cutting device; and further advancing the tissue tunneling assembly towards the cut tissue to advance an inflatable device of the tissue tunneling assembly into the cut tissue to further separate the cut tissue.

In some aspects, the method further includes inflating the inflatable device to further separate the cut tissue.

In some aspects, the inflatable device is wedge shaped, and further advancing the inflatable device into the cut tissue wedges apart the tissue along the cut made by the cutting device.

In some aspects, the method further includes axially moving the inflatable device with respect to the cutting device.

In some aspects, the method further includes selectively sheathing the inflatable device within a sheath and/or unsheathing the inflatable device from within a lumen in the sheath to adjust an inflated length of the inflatable device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of a tissue tunneling system formed in accordance with aspects of the present disclosure.

FIG. 1A illustrates an elevational view of detail area 1A in FIG. 1.

FIG. 1B illustrates a perspective view of detail area 1B in FIG. 1A.

FIG. 1C illustrates an elevational view of an alternate embodiment along the detail area 1A in FIG. 1.

FIG. 2 illustrates an elevational view of a tissue tunneling system formed in accordance with aspects of the present disclosure with an inflatable device movable with respect to a cutting device.

FIG. 3 illustrates an elevational view of a tissue tunneling system formed in accordance with aspects of the present disclosure with an adjustable inflatable device.

FIG. 3A, FIG. 3B, and FIG. 3C illustrate various examples of configurations of an inflatable device in the detail area 3 of FIG. 3.

FIG. 3D illustrates an optional mechanism configured to hold a selected configuration of an inflatable device.

FIG. 4 illustrates an elevational view of a tissue tunneling system formed in accordance with aspects of the present disclosure with a multi-element inflatable device.

FIG. 4A illustrates an example of a configuration of an inflatable device in the detail area 4 of FIG. 4.

FIG. 4B illustrates another example of a configuration of an inflatable device in the detail area 4 of FIG. 4.

FIG. 5 illustrates an example of an embodiment of an alternate configuration of an inflatable device which may be provided in the detail area 1A of FIG. 1.

FIG. 6A illustrates another example of an embodiment of an alternate configuration of an inflatable device which may be provided in the detail area 1A of FIG. 1.

FIG. 6B illustrates an end view of the example of an embodiment of an inflatable device illustrated in FIG. 6A.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. It will be appreciated that reference to “at” the target tissue site is intended to include tissue at and about the vicinity of (e.g., along, adjacent, etc.) the target tissue, and is not limited to just target tissue. As understood herein, corresponding is intended to convey a relationship between components, parts, elements, etc., configured to interact with or to have another intended relationship with one another.

In accordance with various principles of the present disclosure, devices, assemblies, systems, methods, etc., are described for facilitating cutting of tissue, such as tunneling into tissue. For instance, various devices, assemblies, systems, methods, etc., are disclosed which separate tissue, such as to facilitate cutting of the tissue. In some aspects, various devices, assemblies, systems, methods, etc., are disclosed which may impose traction on tissue being cut to facilitate further cutting. In some aspects, various devices, assemblies, systems, methods, etc., of the present disclosure enable blunt dissection of tissue. In some aspects, various devices, assemblies, systems, methods, etc., provide a cutting device in addition to an inflatable device to facilitate cutting of tissue. It will be appreciated that reference may be made herein to medical tools, instruments, devices, etc., interchangeably and without intent to limit. Furthermore, reference may be made interchangeably herein to terms such as cutting, dissecting, resecting, excising, etc., including other grammatical forms thereof, without intent to limit. And, with regard to cutting devices, the present disclosure may refer to surgical knives, such as electrosurgical knives, for the sake of convenience, but other types of cutting devices, such, as without limitation, blades, scalpels, laser-based cutting devices, etc., may be used instead. Such cutting devices may be inserted to a target site through a medical scope such as a gastroscope, endoscope, etc., such as through a working channel thereof, such as to facilitate insertion, navigation, etc., to a target site while protecting the body passage or lumen through which the medical scope is navigated. Reference may be made herein to a target site, anatomical site, treatment site, etc., interchangeably and without intent to limit.

In accordance with various principles of the present disclosure, use of an inflatable device in conjunction with a cutting device enables safer and/or less time intensive procedures such as third space endoscopy procedures. For instance, the cutting device may be used to initiate separation of tissue at a target site, and the inflatable device may then be used to separate the cut tissue. For instance, tissue may be initially cut with the cutting device of the system. Optionally, fluid may be injected into the target site to facilitate such cutting by creating a bleb at the target site prior to cutting. The inflatable device may be inserted within the cut and inflated to exert pressure (e.g., mechanical radial and/or normal forces against the tissue) causing the tissue to be pushed apart, such as may be achieved by a blunt instrument. Upon separating the tissue with the inflatable device, the medical professional may observe the amount/extent of tissue which has been cut to determine if further cutting is medically indicated. For instance, the cutting device and inflatable device may be delivered with a medical scope having a visualization device such as a camera. The scope may be used to visualize the cut area, such as upon separation of the cut tissue by the inflatable device. In some aspects, the inflatable device may be deflated to provide an unobstructed view for the visualization device. In some aspects, the inflatable device is clear, and filled with a clear fluid, to facilitate visualization therethrough even if in the field of view of the visualization device. In some aspects, the inflatable device is deflated to facilitate viewing. Deflation allows visualization of the extent of the separation created by the cutting device and/or inflatable device. If further separation of tissue is medically indicated, the cutting device and/or the inflatable device may be used to further separate the tissue, and visualization may once again be used to assess the extent of tissue separation, such actions being repeated as medically indicated.

The inflatable device is typically positioned proximal to the cutting device. In some aspects, the inflatable device and the cutting device are independently advanceable/retractable with respect to each other and with respect to the target site. In some aspects, the cutting device and the inflatable device are advanced independently of each other to a target site, such as on independent elongate members. In some aspects, the inflatable device is advanced on a tubular elongate member extended over an elongate member with which the cutting device is extended (i.e., with the elongate member which delivers the cutting device extending through the lumen of the tubular elongate member which delivers the inflatable device).

In some aspects, inflation of the inflatable device may be gradual. For instance, in some aspects, a distal portion of the inflatable device is inflated before a proximal portion of the inflatable device is inflated. In some aspects, the inflatable device includes more than one independently inflatable portion so that a distal portion is separately inflatable prior to inflation of a proximal portion. In some aspects, a sheath may be provided over the inflatable device and proximally retracted with respect to the inflatable device (and/or the inflatable device may be distally extended relative to the sheath) to gradually expose an inflated portion of the inflatable device, thereby varying the effective length of the inflatable device. In some aspects, the amount of fluid within the inflatable device affects the pressure within the inflatable device and, consequently, the pressure exerted by the inflatable device on the tissue at the target site.

In some aspects, the inflatable device is a balloon. In some aspects, the inflatable device is formed of a noncompliant material. In some aspects, the inflatable device is inflated with an inflation medium such as a fluid (e.g., saline, water, air, etc.). It will be appreciated that the inflatable device may have any of a variety of sizes, shapes, configurations, and/or dimensions, such selected in view of the nature and various other properties of the target site.

Various embodiments of tissue tunneling devices, assemblies, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements, components, members, etc., similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

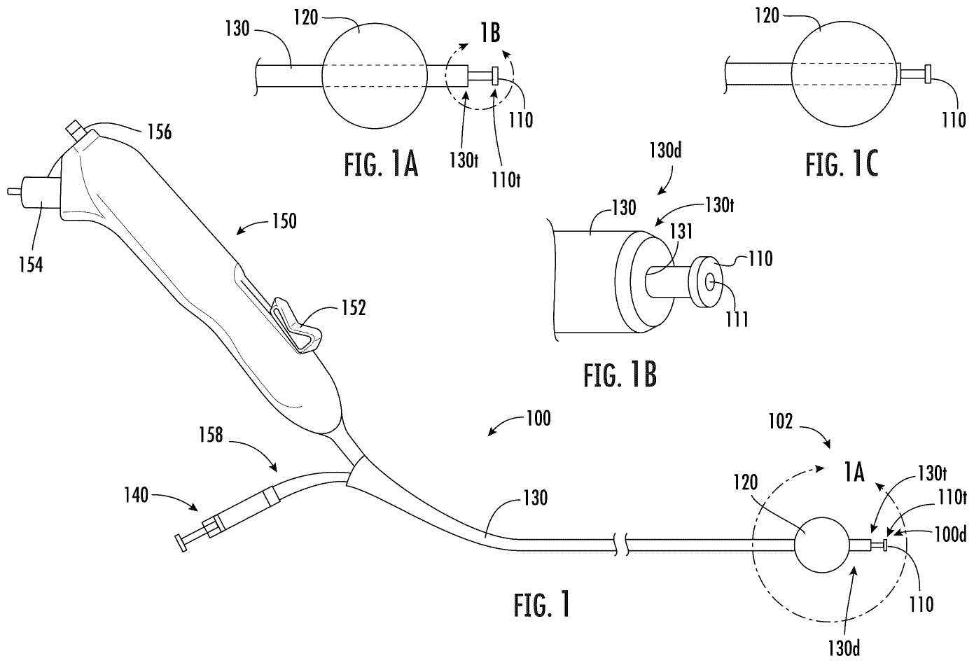

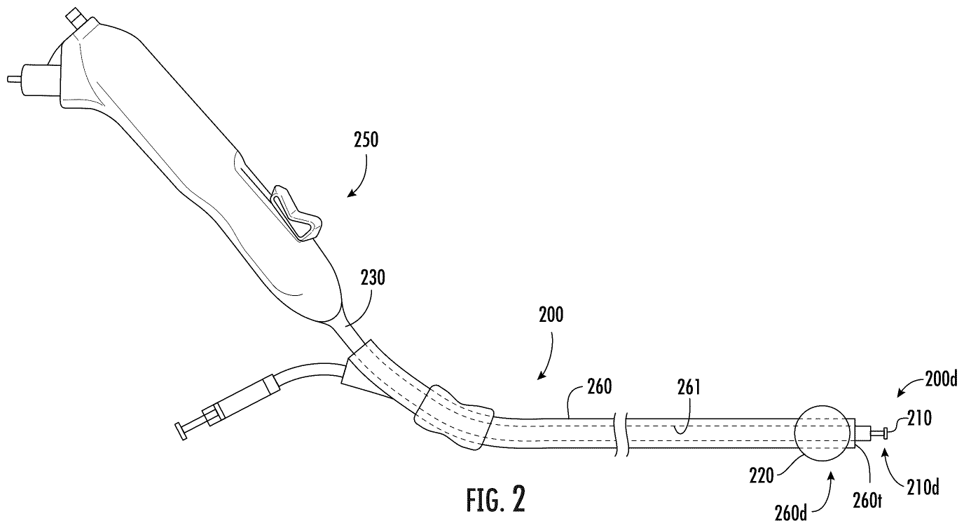

Turning now to the drawings, FIG. 1 illustrates an example of an embodiment of a tissue tunneling system 100 having a tissue tunneling assembly 102 including a cutting device 110 and an inflatable device 120 positioned with respect to each other to separate/tunnel into tissue in accordance with various principles of the present disclosure. In the illustrated example of an embodiment, the inflatable device 120 is positioned proximal to the cutting device 110. As such, the cutting device 110 may be positioned at the target site distal to the cutting device 110 such as to contact tissue at the target site before the inflatable device 120 contacts the tissue. The cutting device 110 is operated/actuated to initiate action of the tissue tunneling system 100 with respect to the target site, such as to cut into the tissue. The inflatable device 120 is advanceable and actuated after the tissue has been cut (or, in some instances, while the tissue is being cut) by the cutting device 110 to further separate the cut tissue. More particularly, once an initial cut in the tissue at the target site has been made by the cutting device 110, the inflatable device 120 may be inserted into the cut created by the cutting device 110, such as by distally advancing the tissue tunneling system 100. The inflatable device 120 may then be expanded/inflated to separate the cut tissue and tunnel further into the target site.

In the example of an embodiment illustrated in FIG. 1, the cutting device 110 and the inflatable device 120 are positioned at the distal end 100d of the tissue tunneling system 100 for advancement to a target site. In some aspects, the distal end 100d of the tissue tunneling system 100 is advanced into a patient's body and to a target site therein through a medical scope, such as an endoscope, gastroscope, etc. (the configuration of which being known to those of ordinary skill in the art, and thus not requiring illustration for an understanding thereof). In the example of an embodiment illustrated in FIG. 1, the cutting device 110 (and optionally also the inflatable device 120) may be advanced with the assistance of an elongate member 130 to a target site. The elongate member 130 may alternately be referenced herein as an elongate delivery member 130 to distinguish over other elongate members described herein. In the example of an embodiment of a tissue tunneling system 100 illustrated in FIG. 1, the cutting device 110 extends distally from a distal end 130d of the elongate member 130 for advancement to the target site, although other arrangements are within the scope and spirit of the present disclosure. In some aspects, the cutting device 110 is operatively coupled to, such as mounted on, the elongate member 130. As shown in FIG. 1A, illustrating, in further detail, area 1A of FIG. 1, and in FIG. 1B, illustrating, in further detail, area 1B of FIG. 1A, in some embodiments, the elongate member 130 has a lumen 131 defined therethrough. In some aspects, the cutting device 110, or at least a proximal portion thereof, is retractable within and extendable out of the lumen 131 of the elongate member 130. For instance, the cutting device 110 may be extended and delivered to a target site retracted, or at least partially retracted, within the lumen 131 of the elongate member 130, to shield the cutting end of the cutting device 110 from the passage through which the cutting device 110 is advanced to the target site, and/or to shield the cutting end of the cutting device 110 when not being used for cutting (e.g., during blunt tissue dissection). The position of the distalmost end 110t of the cutting device 110 may be adjusted with respect to the distalmost end 130t of the elongate member 130 by advancing or retracting the cutting device 110 with respect to (such as through the lumen 131 of) the elongate member 130. For instance, the cutting device 110 may be advanced or retracted with respect to the elongate member 130 to advance the cutting device 110 further into tissue and/or to adjust the length of the cutting portion thereof, such as to adjust various cutting parameters in a manner such as known to those of ordinary skill in the art. If the cutting device 110 is an electrosurgical knife, then electrical connections to the cutting device 110 may extend through the lumen 131 of the elongate member 130 to power the cutting device 110. Although the cutting device 110 is illustrated as a T-shaped electrosurgical knife (e.g., with an electrode configured for precision cutting), the present disclosure need not be limited in this regard.

In some aspects, navigation, steering, positioning, etc., of the cutting device 110 and/or the inflatable device 120 may be controlled via the elongate member 130. The elongate member 130 preferably is sufficiently flexible to navigate through tortuous pathways in a human body. Optionally, the elongate member 130 is configured for advancement through a working channel of an insertion member of a medical scope. In some aspects, navigation, steering, positioning, etc., of the elongate member 130 is controlled via the medical scope through which the elongate member 130 is advanced to a target site.

As noted above, the example of an embodiment of an inflatable device 120 illustrated in FIG. 1 may be operatively associated with the elongate member 130 to be delivered, with the cutting device 110, to a target site. For instance, the inflatable device 120 is illustrated in FIG. 1 and FIG. 1A (illustrating, in further detail, area 1A of FIG. 1) as mounted over a distal end 130d of the elongate member 130. However, other configurations of a tissue tunneling system 100 with an inflatable device 120 delivered to a target site with, or after delivery of, a cutting device 110 are within the scope and spirit of the present disclosure, various examples of alternative embodiments being described in further detail below. As also noted above, the inflatable device 120 of a tissue tunneling system 100 formed in accordance with various principles of the present disclosure is expanded or inflated to separate tissue at the target site, such as tissue which preliminarily has been cut by the cutting device 110 of the tissue tunneling system 100. Inflation of the inflatable device 120 may be controlled by the medical professional to establish a size (e.g., cross-sectional size, such as a diameter or cross-sectional area) of the inflatable device 120 and optionally an associated pressure exerted by the inflatable device 120 to achieve the desired separation of tissue at the target site. In some aspects, the tissue tunneling system 100 includes an inflatable device controller 140 fluidly coupled with the inflatable device 120, such as via a lumen through the elongate member 130 (such as in instances in which the inflatable device 120 is operatively coupled with the elongate member 130 for delivery therewith to a target site). Additionally or alternatively, a separately-formed lumen (e.g., extending along the elongate member 130) may fluidly couple the inflatable device controller 140 and the inflatable device 120. In the example of an embodiment illustrated in FIG. 1, the inflatable device controller 140 is illustrated as a syringe with an inflatable medium (e.g., a fluid) with which the inflatable device may be filled upon actuation of the inflatable device controller 140. However, it will be appreciated that other configurations of inflatable device controllers, such as other forms of fluid sources or insufflation devices (e.g., with manual or automated fluid pumps and/or with a pressure gauge), are within the scope and spirit of the present disclosure.

In some aspects, in addition to being fluidly coupled with a fluid-delivering device or system to deliver an inflation fluid to the inflatable device 120, the tissue tunneling system 100 may also deliver a fluid to the target site to form a bleb at the target site to facilitate cutting by the cutting device 110. In some aspects, a fluid (e.g., water, saline, air, etc.) is delivered and injected into the target site via a lumen 131 defined through the elongate member 130 with which the cutting device 110 is delivered. The fluid may be injected into the target site to create a bleb to facilitate cutting by the cutting device 110. In some aspects, such as illustrated in FIG. 1B, the lumen 131 is fluidly coupled with a lumen 111 extending through the cutting device 110, and the injected fluid is delivered via the cutting device 110.

As noted above, once the cutting device 110 has been activated to cut tissue at the target site, the inflatable device 120 may be inserted into the separated/cut area of tissue. As such, the inflatable device 120 may be a blunt cutting instrument of the tissue tunneling system 100, capable of performing blunt tissue dissection. The inflatable device 120 may initially be inserted in a deflated or at least partially deflated configuration, and then inflated (e.g., gradually inflated) to move apart the tissue along the cut created by the cutting device 110. The medical professional may assess the target site to determine if further cutting is medically indicated for the procedure being performed. For instance, a visualization instrument (e.g., a camera) may be inserted to view the cut area, such as with the inflatable device 120 holding apart the already-separated tissue layers. In some aspects, the inflatable device 120 is delivered to the target site with the use of a medical scope, such as described above, and the visualization instrument is a visualization element associated with the medical scope (e.g., a camera, optical fiber, etc., extending through, such as a component of, the medical scope). Optionally, the inflatable device 120 may be further inflated to further separate the tissue which has been initially separated by the cutting device 110.

A tissue tunneling system 100 formed in accordance with various principles of the present disclosure optionally includes a control handle 150 at the proximal end 100p thereof, such as illustrated in FIG. 1. The illustrated example of an embodiment of a control handle 150 is operatively associated with the cutting device 110 and/or the inflatable device 120, and may be grasped in the hand of a medical professional while operating the tissue tunneling system 100. Movements of the control handle 150 may be transferred to one or more members/elements of the tissue tunneling system 100, such as to manipulate the position of the tissue tunneling system 100 with respect to a target site. In some aspects, the control handle 150 is operatively associated with the cutting device 110 and/or the inflatable device 120 so that a medical professional may control operation of the cutting device 110 and/or the inflatable device 120 via the control handle 150.

The example of an embodiment of a control handle 150 illustrated in FIG. 1 includes a cutting device controller 152 operatively associated with the cutting device 110. The illustrated example of an embodiment of a cutting device controller 152 is movable with respect to the control handle 150 to move the cutting device 110 at the distal end 100d of the tissue tunneling system 100. In some embodiments, the cutting device controller 152 is slidable with respect to the control handle 150. For instance, in some embodiments, the cutting device controller 152 is advanceable distally to advance the cutting device 110 distally, and retractable proximally to retract the cutting device 110 proximally. In some aspects, distal and proximal movement of the cutting device 110 may be with respect to the distalmost end of the elongate member 130, such to adjust the length of the cutting device 110 which extends distal to the distalmost end 130t of the elongate delivery member 130. As may be appreciated by those of ordinary skill in the art, modifications to the cutting device 110 may be desirable such as to adjust various cutting parameters (e.g., the length of the cutting element; the magnitude of energy supplied to a powered cutting device, such as an electrosurgical knife, to adjust the energy delivery to tissue; etc.).

In embodiments in which the cutting device 110 is an electrosurgical knife, the control handle 150 may include an electrical connector 154 configured to electrically couple the cutting device 110 with a power source (such as known to those of ordinary skill in the art and thus not warranting detailed illustration thereof). In embodiments in which a fluid is injected into the target site (such as to create a bleb to facilitate cutting), a fluid port 156 may be provided along the control handle 150. The fluid port 156 may be fluidly coupled with a lumen 131 through the elongate member 130 (such as illustrated in FIG. 1B). For instance, in some embodiments, the fluid port 156 is fluidly coupled with a lumen 131 of the elongate member 130 for delivery out the distal end 130d of the elongate member 130 to a target site. In some aspects, fluid may be delivered through the lumen 131 of the elongate member 130 with the cutting device 110. Optionally, fluid is delivered through a lumen 111 defined through the cutting device 110.

In some aspects, the above-described inflatable device controller 140 is operatively associated with the control handle 150. For instance, the example of an embodiment of a control handle 150 illustrated in FIG. 1 includes a port 158 with which the inflatable device controller 140 may be fluidly couple to deliver fluid to the inflatable device 120. In some aspects, an inflation medium is delivered to the inflatable device 120 via a lumen through the elongate member 130 (e.g., through the lumen 131 through which the cutting device 110 extends), or through a separate tubular elongate member extending along or through the elongate member 130.

As noted above, the inflatable device 120 is delivered to a target site proximal to the cutting device 110. In the example of an embodiment illustrated in FIG. 1, the inflatable device 120 is mounted on the distal end 130d of the elongate member 130 through which the cutting device 110 may be advanced (as described above), and thus proximal to the cutting device 110. As illustrated in area A of FIG. 1, and in further detail in FIG. 1A, the inflatable device 120 may be mounted proximally spaced from the distalmost end 130t of the elongate member 130 and thus proximally spaced from the cutting device 110. However, other arrangements are within the scope and spirit of the present disclosure. For instance, as illustrated in FIG. 1C, showing a modified arrangement of elements within area 1A of a tissue tunneling system 100 such as the illustrated in FIG. 1, an inflatable device 120 of a tissue tunneling system 100 formed in accordance with various principles of the present disclosure may be positioned closer to the cutting device 110 than as illustrated in FIG. 1A, such as leaving little gap therebetween. For instance, the inflatable device 120 can be as close as 0-2 cm from the distalmost end 130t of the elongate delivery member 130.

In accordance with various principles of the present disclosure, the cutting device and the inflatable device of a tissue tunneling system formed in accordance with various principles of the present disclosure need not be fixed with respect to each other. Additionally or alternatively, the cutting device and the inflatable device need not be operatively associated with the same elongate member for delivery to the target site. For instance, the cutting device of a tissue tunneling system formed in accordance with various principles of the present disclosure may delivered with a first elongate member, while the inflatable device of the system may be delivered separately, such as with a second elongate member. As such, the inflatable device need not be fixed with respect to the cutting device or with respect to the elongate member with which the cutting device is operatively associated (e.g., fixedly or otherwise coupled or mounted to for delivery to a target site), and the axial distance between the cutting device and the inflatable device may thus be adjustable. For instance, in the example of an embodiment of a tissue tunneling system 200 illustrated in FIG. 2, the inflatable device 220 is mounted on a separate elongate member 260 advanceable to the target site independently of the elongate member 230 with or on which the cutting device 210 is advanced to the target site. As such, cutting device 210 and the first elongate member 230 are separate and independently movable with respect to the inflatable device 220 and the second elongate member 260. In some aspects, the second elongate member 260 is a tubular elongate member with a lumen 261 defined therethrough. In some aspects, the lumen 261 of the second elongate member 260 is sized for the first elongate member 230 to extend therethrough. As such, the cutting device 210 may be delivered to a target site within the distal end 260d of the second elongate member 260, with the second elongate member 260 configured as a sheath over the distal end 210d of the cutting device 210. In some aspects, the second elongate member 260 is sized to extend through a further tubular elongate delivery member, such as the working channel of a medical scope, such as described above with reference to the example of an embodiment of a tissue tunneling system 100 illustrated in FIG. 1. It will be appreciated that various other elements, members, assemblies, devices, etc., of the tissue tunneling system 200 illustrated in FIG. 2 which are similar to or substantially the same as elements in the tissue tunneling system 100 illustrated in FIG. 1 are indicated with the same reference characters increased by 100, reference being made to descriptions thereof provided with respect to FIG. 1 for the sake of brevity.

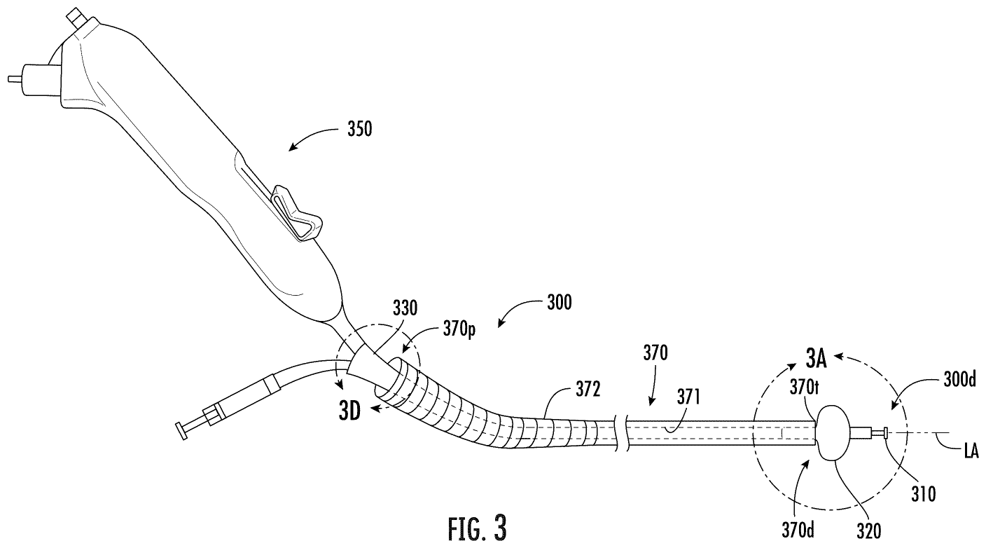

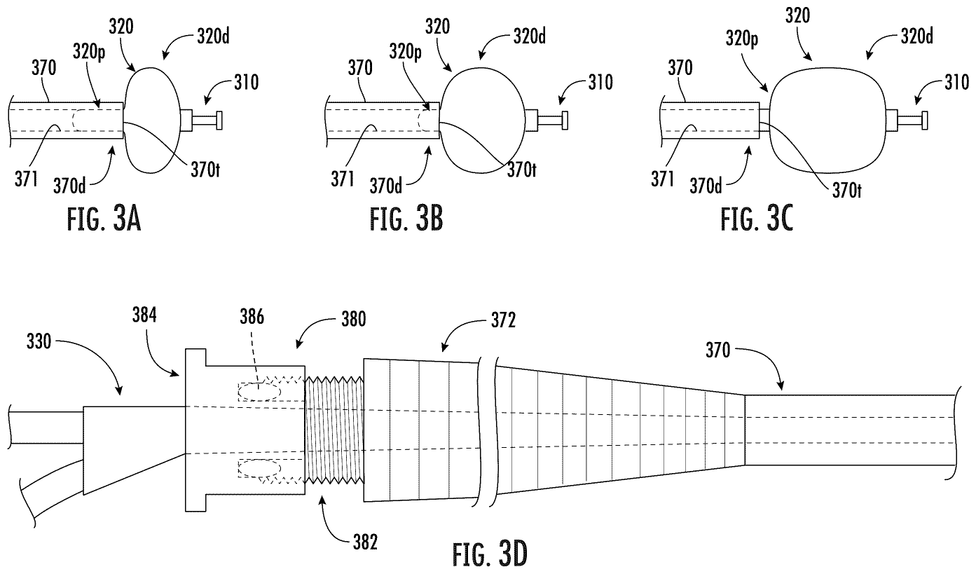

As noted above, an inflatable device of a tissue tunneling system formed in accordance with various principles of the present disclosure may be inflatable to different diameters. In order to gradually increase the distance between tissue layers at the target site (e.g., at a cut created by a cutting device of the present disclosure), an inflatable device of the present disclosure may be gradually inflated. In some aspects, in addition to or instead of being gradually inflated, a sheath 370 may be provided over an inflatable device 320 formed in accordance with various principles of the present disclosure to regulate inflation thereof within a cut tissue, such as in the example of an embodiment of a tissue tunneling system 300 illustrated in FIG. 3.

More particularly, the example of an embodiment of a tissue tunneling system 300 illustrated in FIG. 3 includes a tubular elongate member 370 having a distal end 370d movable over and with respect to an inflatable device 320, which, in turn, is positioned proximal to a cutting device 310. The tubular elongate member 370 has a lumen 371 defined axially therethrough sized to fit the inflatable device 320 therein. For the sake of convenience, and without intent to limit, the tubular elongate member 370 may be referenced herein as a sheath 370 to differentiate from other tubular elongate members of a tissue tunneling system formed in accordance with various principles of the present disclosure. In some aspects, the inflatable device 320 may be delivered to a target site within the distal end 370d of the sheath 370. In some aspects, the inflatable device 320 is delivered distal to the distal end 370d of the sheath 370, and the sheath 370 is advanceable distally over the inflatable device 320 to regulate the inflation/inflation size of the inflatable device 320. More particularly, the sheath 370 may be positioned over the inflatable device 320 a selected axial extent (e.g., along the length of the inflatable device 320 such as along the longitudinal axis of the tissue tunneling system 300) which affects the axial extent/length of an inflated portion of the inflatable device 320 which is not confined or restrained or constrained within the lumen 371 of the sheath 370 (e.g., the axial extent of the inflatable device 320 which extends distally beyond the distal end 370d of the sheath 370). The sheath 370 may be distally advanceable and/or proximally retractable with respect to the inflatable device 320 to adjust the length of the inflated portion of the inflatable device 320. Such adjustment of the length of the inflatable portion of the inflatable device 320 may be advantageous in affecting the amount of tissue separated by the inflatable device 320 inserted and inflated therebetween.

In some embodiments, a grip 372 (e.g., a wider diameter portion; a portion covered with gripping material such as cushioned and/or non-slip material; a portion textured for comfort and/or to reduce slippage when manually grasped; etc.) is provided at a proximal end 370p of the sheath 370, such as to facilitate grasping by a medical professional and/or connection with another controller of the sheath 370 (e.g., automated controller). In some aspects, the sheath 370 is axially movable with respect to, such as to axially-movably extend over, an elongate member 330 which may be used to deliver the cutting device 310 to a target site. For instance, a cutting device 310 of a tissue tunneling system 300 such as illustrated in FIG. 3 may be operatively associated with (e.g., mounted on a distal end 330d of) an elongate member 330 and advanced, with the distal end 330d of the elongate member 330, to a target site. The inflatable device 320 is positioned proximal to the cutting device 310 for delivery to the target site as well. It will be appreciated that the inflatable device 320 may be operatively associated with (e.g., mounted on) the elongate member 330 or on a separate and independent second elongate member such as described above with respect to the example of an embodiment of a tissue tunneling system 200 illustrated in FIG. 2.

In use, the cutting device 310 may first be actuated to create an initial cut at the target site. The inflatable device 320 may then be inserted, at least partially, into the cut area of tissue. In some aspects, as noted above, the inflatable device 320 may be delivered to a target site while within (e.g., fully within) the lumen 371 of the sheath 370. The inflatable device 320 may be extended into the cut area either within the sheath 370, or with at least a portion thereof distally extended out of the sheath 370, beyond the distalmost end 370t of the sheath 370. In accordance with various principles of the present disclosure, the sheath 370 is movable (e.g., axially movable) with respect to the inflatable device 320. More particularly, the sheath 370 is proximally retractable and/or the inflatable device 320 is distally advanceable with respect to the inflatable device 320 to expose/uncover a selected length of the inflatable device 320. As the inflatable device 320 is inflated, the sheath 370 may be retracted proximally and/or advanced distally with respect to the inflatable device 320 to vary the portion/length of the inflatable device 320 extended outside the sheath 370. The portion/length of the inflatable device 320 outside the sheath 370 is unconstrained by the sheath 370, and thus able to be inflated to a desired (e.g., up to a maximum) cross-sectional size (transverse to the axial length of inflatable device 320) for at least such portion of the inflatable device 320 to separate tissue at the target site. As may be appreciated, the position of the sheath 370 relative to the inflatable device 320 as the inflatable device 320 is inflated affects distribution (and optionally also the pressure) of the inflation fluid within the inflatable device 320.

Various positions of a sheath 370 with respect to an inflatable device 320, such as within area 3 of a tissue tunneling system 300 as illustrated in FIG. 3, are illustrated in FIGS. 3A-3C. As may be appreciated, the position of the sheath 370 relative to the inflatable device 320 determines/controls the length of the inflatable device 320 which may be inflated. As illustrated in FIG. 3A, the sheath 370 covers a proximal portion 320p of the inflatable device 320 and constrains inflation of such proximal portion 320p. As such, a distal portion 320d of the inflatable device 320 is inflated to a greater extent than the proximal portion 320p may be inflated. As the sheath 370 is moved further proximally with respect to the inflatable device 320, more of the inflatable device 320 is advanced out of the lumen 371 of the sheath 370. As illustrated in FIG. 3B, a greater extent of a distal portion 320d of the inflatable device 320 than as illustrated in FIG. 3A has extended outside the lumen 371 of the sheath 370, and a smaller proximal portion 320p remains within the lumen 371. In some aspects, the sheath 370 may be fully retracted from sheathing the inflatable device 320 to allow unrestricted inflation (along the full length/extent) of the inflatable device 320, such as illustrated in FIG. 3C. In some aspects, FIGS. 3A-3C may be considered to illustrate sequential stages of retraction of a sheath 370 with respect to an inflatable device 320. In some aspects, the sheath 370 may be proximally retracted and then distally extended during the same procedure, such as depending on conditions at the target site.

Optionally, a tissue tunneling system 300 such as illustrated in FIG. 3 includes a mechanism 380 configured to hold the sheath 370 in place with respect to the inflatable device 320, such as to maintain a desired extent the inflatable device 320 extends out of the sheath 370. In some aspects, such mechanism 380 is positioned and/or mounted adjacent or with respect to the grip 372. An example of an embodiment of a holding mechanism 380 is illustrated in FIG. 3D as a Tuohy-Borst-like adaptor mechanism 380 that may hold the sheath 370 in place (e.g., with respect to the elongate member 330) when the mechanism 380 is tightened. For instance, the sheath 370 may include an externally-threaded extension 382 over which an internally-threaded collar 384 is positioned. Similar to a Tuohy-Borst adaptor, rotation of the collar 384 with respect to the extension 382 may cause a gasket 386 within the collar 384 (and mounted over the elongate member 330) to tighten the gasket 386 over the elongate member 330. It will be appreciated that other appropriate mechanisms known to those of ordinary skill in the art, such as a collet-type configuration, a clamp, etc., may be used instead of a Tuohy-Borst type configuration. The user may thereby set/lock the inflation length of the inflatable device 320 to a desired extent.

It will be appreciated that the provision of a sheath 370 such as illustrated in FIG. 3 may advantageously allow the use of longer balloons than may be initially considered for use in a given situation. Depending on the clinical scenario, if the user prefers to use only a few millimeters of balloon length for tissue separation, the remainder of the inflatable device 320 may remain within the lumen 371 of the sheath 370 without affecting the target site. As additional separation of tissue is determined to be medically indicated, the sheath 370 may be progressively retracted to increase the length of the inflatable device 320 which is inflated and configured to separate tissue at the target site.

It will further be appreciated that the use of a sheath 370 may advantageously limit the effect of inflation of an inflatable device 320 on another tubular elongate delivery member (e.g., a medical scope) through which the inflatable device 320 may be delivered to the target site (e.g., as described above). For instance, inflation of the proximal portion 320p of the inflatable device 320 may be restrained by the sheath 370. And, the relative positions of the distal end 370d of the sheath 370 and the distal end of the tubular elongate delivery member may be adjusted to adjust inflation of the inflatable device 320 relative to the tubular elongate delivery member. As such, inflation of the inflatable device 320 need not commence within the tubular elongate delivery member (which could cause proximal movement of the tubular elongate delivery member as the inflatable device 320 expands).

It will be appreciated that a sheath 370 as illustrated in FIG. 3 to adjust the inflatable device thereof may be included in other embodiment of tissue tunneling systems formed in accordance with various principles of the present disclosure, such as, without limitation, the examples of embodiments of tissue tunneling systems 100 and 200 illustrated in FIG. 1 and FIG. 2, respectively. Moreover, it will be appreciated that various other elements, members, assemblies, devices, etc., of the tissue tunneling system 300 illustrated in FIG. 3 which are similar to or substantially the same as elements in the tissue tunneling system 100 illustrated in FIG. 1 are indicated with the same reference characters increased by 200, reference being made to descriptions thereof provided with respect to FIG. 1 for the sake of brevity.

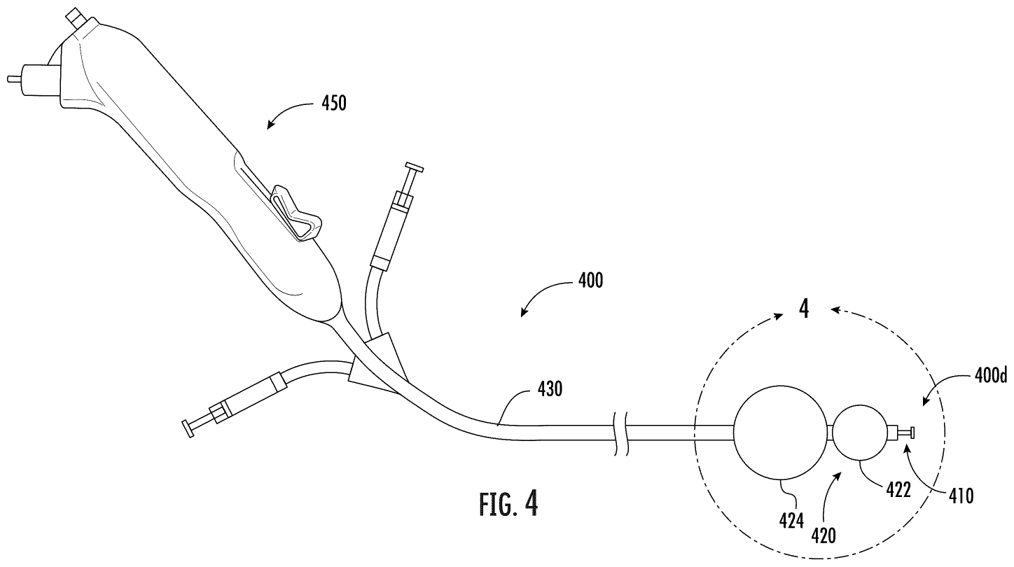

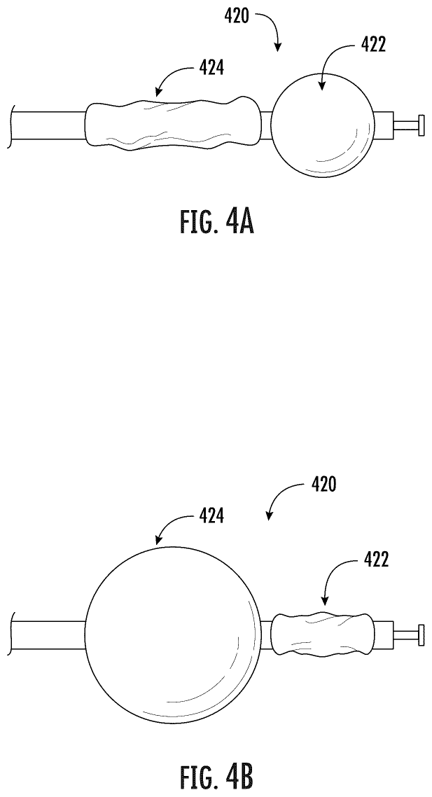

In accordance with various principles of the present disclosure, a tissue tunneling system may have more than one inflatable device configured to separate tissue at a target site, such as after cutting tissue with a cutting device of the tissue tunneling system. The provision of more than one inflatable device may allow for further variability and/or precision in separating tissue at a target site. For instance, in the example of an embodiment of a tissue tunneling system 400 illustrated in FIG. 4, the inflatable device 420 includes a first inflatable element 422, and a second inflatable element 424 positioned proximal to the first inflatable device 422. For the sake of convenience, and without intent to limit, reference is made to two inflatable elements 422, 424 of a multi-element inflatable device 420 formed in accordance with various principles of the present disclosure. However, it will be appreciated that additional inflatable elements may be provided (i.e., the inflatable device may have three or more inflatable elements) for further adjustability of the tissue tunneling system, such as when used for blunt tissue dissection. Reference may thus be made to a multi-element inflatable device 420 as having two or more inflatable elements which may be considered to be structurally distinct from one another (yet which may be considered part of an inflatable device assembly generally referenced herein as a multi-element inflatable device 420).

In some embodiments, the inflatable elements 422, 424 of a multi-element inflatable device 420 formed in accordance with various principles of the present disclosure are independently inflatable. In some embodiments, the inflatable elements 422, 424 of the multi-element inflatable device 420 are different sizes. In the example of an embodiment illustrated in FIG. 4, the distal inflatable element 422 has a diameter smaller than the diameter of the proximal inflatable element 424. However, a reverse configuration is within the scope of the present disclosure as well. In some aspects, the inflatable elements 422, 424 of a multi-element inflatable device 420 of a tissue tunneling system 400 may be sequentially inflated. For instance, the distal inflatable element 422 may be inflated at a target site before the proximal inflatable element 424 is inflated, such as shown in FIG. 4A, schematically illustrating in further detail an example of a configuration of area 4 of a tissue tunneling system 400 such as illustrated in FIG. 4. The proximal inflatable element 424 may be inflated after tissue has been separated by the distal inflatable element 422 and a determination has been made if further inflation of the proximal inflatable device 424 is medically indicated. In some aspects, the distal inflatable element 422 and may remain inflated as the proximal inflatable element 424 is inflated, such as represented in area A in FIG. 4, such to increase the separation of tissue at a target site. In some aspects, the distal inflatable element 422 may be deflated while or after the proximal inflatable element 424 is inflated, such as shown in FIG. 4B, schematically illustrating an example of a configuration of detail area 4 of a tissue tunneling system 400 such as FIG. 4. Various other combinations of orders and/or degrees of inflation of elements of a multi-element inflatable device 420 are within the scope and spirit of the present disclosure.

It will be appreciated that a multi-element inflatable device 420 such as illustrated in FIG. 4 may be included in other embodiments of tissue tunneling systems formed in accordance with various principles of the present disclosure, such as, without limitation, the examples of embodiments of tissue tunneling systems 100, 200, and 300, illustrated, respectively, in FIG. 1, FIG. 2, and FIG. 3. Moreover, it will be appreciated that various other elements, members, assemblies, devices, etc., of the tissue tunneling system 400 illustrated in FIG. 4 which are similar to or substantially the same as elements in the tissue tunneling system 100 illustrated in FIG. 1 are indicated with the same reference characters increased by 300, reference being made to descriptions thereof provided with respect to FIG. 1 for the sake of brevity.

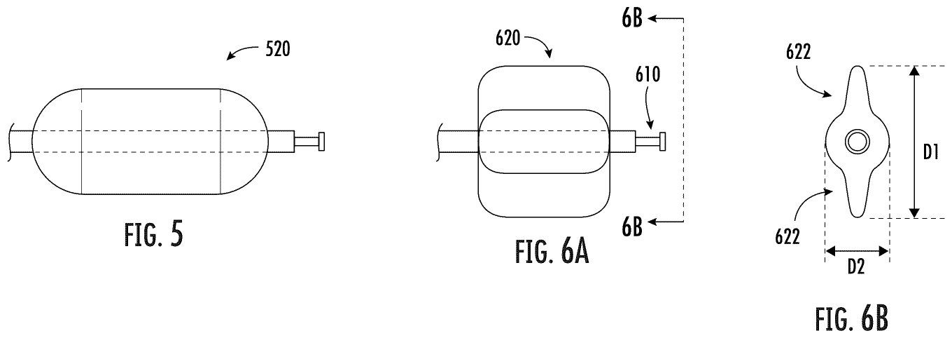

It will be appreciated that the inflatable devices illustrated in the accompanying drawings are simply examples, and may even be considered schematic. The inflatable devices 120, 220, 320, 420 need not have shapes and/or configurations exactly as illustrated. Thus, although illustrated as substantially spherical and/or with a substantially circular cross-sectional shape, an inflatable device of a tissue tunneling system formed in accordance with various principles of the present disclosure may have an other-than-spherical shape/configuration. For instance, a tissue tunneling system formed in accordance with various principles of the present disclosure may have a generally longitudinally-elongated inflatable device 520, such as generally schematically illustrated in FIG. 5 (e.g., showing an alternative configuration of area 1 in FIG. 1). Additionally or alternatively, it will be appreciated that the inflatable device of a tissue tunneling system formed in accordance with various principles of the present disclosure need not have a symmetrical cross-sectional shape. For instance, an inflatable device of a tissue tunneling system formed in accordance with various principles of the present disclosure may have a partially symmetrical configuration (e.g., along only one axis), or may be asymmetrical. Additionally or alternatively, the cross-sectional shape of an inflatable device of a tissue tunneling system formed in accordance with various principles of the present disclosure may be configured to facilitate insertion of the inflatable device into tissue preliminarily cut by the cutting device positioned distal to the inflatable device of the tissue tunneling system. For instance, an example of an embodiment of a partially symmetrical inflatable device 620 is illustrated in FIG. 6A (e.g., showing another alternative configuration of area 1 in FIG. 1). In some aspects, the partially symmetrical inflatable device 620 has a generally elongated cross-sectional shape, with a first cross-sectional dimension D1 larger than a second cross-sectional dimension D2 transverse to (e.g., perpendicular to) the first cross-sectional dimension D1, such as illustrated in FIG. 6B. In some aspects, the cross-section of the partially symmetrical inflatable device 620 may be oblong. In some aspects, the cross-sectional shape of the partially symmetrical inflatable device 620 has narrower sides, such as formed by side extensions or wings 622, causing the partially symmetrical inflatable device 620 to have a larger first cross-sectional dimension D1 along the direction in which the wings 622 extend. In some aspects, wings 622 such as illustrated in FIG. 6B may facilitate insertion into narrow cuts in tissue at the target site, such by presenting a relatively flat portion of the inflatable device 620 for insertion into a cut initially created by the cutting device 610. The widening of the partially symmetrical inflatable device 620 may allow the tissue to be wedged apart as the wider part is further inserted into the cut. Rotation of the partially symmetrical inflatable device 620 may further separate the tissue at the cut. Various other configurations than multiple elements of an inflatable device and/or asymmetrical cross-sections may be used to wedge apart tissue at a target site in accordance with various principles of the present disclosure. For instance, the shape of an inflatable device formed in accordance with various principles of the present disclosure may taper distally (e.g., may have a generally conical or wedge shape) to gradually increase the size of the inflatable device as it is distally inserted between cut tissue at a target site. It will be appreciated that inflatable devices may be formed in accordance with various principles of the present disclosure with various other shapes and configurations than those described above without detracting from the scope and spirit of the present disclosure.

It is to be understood by one of ordinary skill in the art that the present discussion is a description of illustrative examples of embodiments only, and is not intended as limiting the broader aspects of the present disclosure. Thus, although inflatable devices as disclosed herein are described as being used with a cutting device such as an electrosurgical knife, other uses of such inflatable devices are contemplated. It will further be appreciated that the concepts disclosed herein may be particularly suitable for use with peroral endoscopic myotomy (POEM) procedures, recanalization for complete esophageal obstruction (per-oral endoscopic tunneling for restoration of the esophagus), endoscopic submucosal dissection (ESD), submucosal tunneling endoscopic resection (STER), Zenker's Diverticulotomy (ZPOEM), endoscopic gastric pyloromyotomy (GPOEM), per-rectal endoscopic myotomy, diverticular POEM, tunneling recanalization of the esophagus (POETRE). and other third space endoscopic procedures, such as used for treating early cancers in the gastrointestinal tract. However, the concepts disclosed herein may have broader applications as well.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue tunneling system having a proximal end and a distal end, said tissue tunneling system comprising:
a control handle at the proximal end of the tissue tunneling system;
an elongate delivery member extending distally from said control handle;
a cutting device operatively associated with said elongate delivery member for delivery at the distal end of said tissue tunneling system; and
an inflatable device operatively associated with the distal end of said tissue tunneling system.

2. The tissue tunneling system of claim 1, wherein said inflatable device is operatively associated with the distal end of said tissue tunneling system to be delivered to a target site proximal to said cutting device.

3. The tissue tunneling system of claim 2, wherein said inflatable device is axially movable with respect to said cutting device.

4. The tissue tunneling system of claim 3, wherein said inflatable device is operatively associated with a tubular elongate member axially shiftable with respect to said elongate delivery member.

5. The tissue tunneling system of claim 4, wherein said tubular elongate member defines a lumen therethrough in fluid communication with said inflatable device to deliver an inflation medium to said inflatable device to selectively expand said inflatable device.

6. The tissue tunneling system of claim 4, wherein said tubular elongate member defines a lumen therethrough and said elongate delivery member is insertable and axially translatable through the lumen of said tubular elongate member to move said cutting device and said inflatable device with respect to each other.

7. The tissue tunneling system of claim 1, wherein, in addition to being inflatable, the configuration of said inflatable device is adjustable.

8. The tissue tunneling system of claim 7, further comprising a sheath defining a lumen therethrough sized for said inflatable device to fit therein, said sheath advanceable and retractable with respect to said inflatable device to extend over or to unsheathe said inflatable device.

9. The tissue tunneling system of claim 1, wherein said inflatable device comprises two or more inflatable elements.

10. The tissue tunneling system of claim 1, wherein said control handle comprises a first lumen for injection of fluid through a lumen defined through said cutting device, and a second lumen for injection of inflation fluid into said inflatable device.

11. A tissue tunneling assembly comprising:
a cutting device at a distal end of said tissue tunneling assembly; and
an inflatable device positioned proximal to said cutting device.

12. The tissue tunneling assembly of claim 11, wherein said inflatable device is axially movable with respect to said cutting device.

13. The tissue tunneling assembly of claim 11, further comprising a sheath with a lumen therethrough, wherein said sheath is axially movable with respect to said inflatable device and said inflatable device and the lumen are sized for the inflatable device to be selectively fully or partially sheathed within the lumen, partially extended and inflated outside the lumen, or fully extended and inflated outside the lumen.

14. The tissue tunneling assembly of claim 11, wherein said inflatable device comprises two or more separate independently inflatable elements.

15. The tissue tunneling assembly of claim 11, wherein said cutting device is an electrosurgical knife.

16. A method of tunneling into tissue, said method comprising:
advancing a tissue tunneling assembly with a cutting device to a target site;
cutting tissue with the cutting device; and
further advancing the tissue tunneling assembly towards the cut tissue to advance an inflatable device of the tissue tunneling assembly into the cut tissue to further separate the cut tissue.

17. The method of claim 16, wherein further comprising inflating the inflatable device to further separate the cut tissue.

18. The method of claim 16, wherein the inflatable device is wedge shaped, and further advancing the inflatable device into the cut tissue wedges apart the tissue along the cut made by the cutting device.

19. The method of claim 16, further comprising axially moving the inflatable device with respect to the cutting device.

20. The method of claim 16, further comprising selectively sheathing the inflatable device within a sheath and/or unsheathing the inflatable device from within a lumen in the sheath to adjust an inflated length of the inflatable device.
